(19) United States
(12) Patent Application Publication
(10) Pub. No.: US 2026/0098065 A1
(43) Pub. Date: Apr. 9, 2026

(54) METHOD FOR ATTENUATING EOSINOPHILS-DRIVEN INFLAMMATION USING A COMPOSITION FOR MUCOSAL ADMINISTRATION

(71) Applicant: ADVAGENE BIOPHARMA CO., LTD., Taipei City (TW)

(72) Inventors: Han-Pin KUO, TAIPEI CITY (TW); Yu-Shen HSU, TAIPEI CITY (TW); Ming-I CHANG, TAIPEI CITY (TW); Yi-Shiuh HWANG, TAIPEI CITY (TW)

(73) Assignee: ADVAGENE BIOPHARMA CO., LTD., Taipei City (TW)

(21) Appl. No.: 19/341,203

(22) Filed: Sep. 26, 2025

Related U.S. Application Data
(60) Provisional application No. 63/704,305, filed on Oct. 7, 2024.

(51) Int. Cl. C07K 14/245; A61K 38/00; A61P 11/06

(52) U.S. Cl. CPC C07K 14/245; A61P 11/06; A61K 38/00

(57) ABSTRACT

The present disclosure provides a method for treating a subject having eosinophil-associated inflammation, such as severe asthma, comprising administering to the subject a therapeutically effective amount of a composition comprising detoxified Escherichia coli labile toxin (LT).

### FIELD OF THE INVENTION

The invention relates to a method for attenuating eosinophils-driven inflammation and targeting the circulation, airway mucosa, and epithelium-associated cavities. Particularly, the invention relates to a method for treating syndromes associated with excessive eosinophils, including but not limited to eosinophilic asthma, eosinophilic chronic rhinosinusitis (eCRS), and eosinophilic esophagitis (EoE), through intranasal or mucosal administration of a composition containing a genetically detoxified Escherichia coli heat-labile toxin (LT), particularly LTh(αK).

### BACKGROUND OF THE INVENTION

Eosinophils are a type of pro-inflammatory white blood cell that plays a crucial role in mucosal defenses and immune responses against parasitic and viral infections. Eosinophils comprise less than 4% of the white blood cell population in healthy persons. However, elevated eosinophils in type 2 inflammatory illness, along with cytokines such as IL-13, IL-4, and IL-5 from Th2 cells and innate lymphoid cells (ILC2), are commonly observed. Th2 cytokine elevations contribute to the diseases, including but not limited to uncontrolled eosinophilic asthma, eosinophilic rhinosinusitis (cCRS), eosinophilic granulomatosis with polyangiitis, hyper-eosinophilic syndromes, and chronic obstructive pulmonary disease.

Clinically, uncontrolled asthma and eCRS are predominantly chronic eosinophilic diseases. Among patients with uncontrolled asthma or eCRS, elevated eosinophil counts in blood or tissues are observed in approximately 40% to 66% of cases. In asthma, blood eosinophil counts exceeding 300 cells per microliter (μL) are frequently associated with worse asthma control and a higher risk of exacerbations. Chronic rhinosinusitis with nasal polyps (CRSwNP) represents a more progressive form of eCRS characterized by bilateral, endoscopically visualized polyps in the middle meatus. In CRSwNP patients, elevated eosinophil counts in blood or tissue are strongly associated with increased disease severity, nasal polyp (NP) recurrence, impaired olfaction, and a greater need for repeat sinonasal surgery. The severity of eosinophilia has been classified based on absolute eosinophil count (AEC) into mild (upper limit of normal to 1.5×10\hat{ }⁹/L), moderate (1.5-5×10\hat{ }⁹/L), and severe (>5×10\hat{ }⁹/L). Under normal conditions, eosinophils constitute less than 3% of the leukocytes in sputum. Eosinophil counts above this threshold in sputum indicate eosinophilic airway inflammation (J Allergy Clin Immunol Pract 2023; 11:2630-41).

Eosinophil-associated diseases, including but not limited to chronic severe asthma and eCRS, are often resistant to high-dose inhaled corticosteroids (ICS), and they frequently require oral corticosteroids (OCS) to manage symptoms during exacerbations. Despite these treatments, many patients continue to experience inadequate symptom control, resulting in a poor quality of life, frequent disease flare-ups, progressive loss of lung function, and an increased risk of medication-related side effects.

Therapies targeting Th2 cytokines such as IL-13, IL-4, and IL-5, along with their corresponding receptors and thymic stromal lymphopoietin (TSLP), have shown promise in reducing eosinophil counts and managing inflammation and exacerbations in patients with asthma. Despite the effectiveness of biologics in treating airway infiltrative eosinophils, their high cost has limited accessibility for a significant portion of patients, underscoring the unmet medical demand for more accessible and effective therapies (Lambrecht B N, Hammad H, Fahy J V: The Cytokines of Asthma. Immunity 2019, 50(4):975-991).

Eosinophilic uncontrolled asthma is a sub-phenotypic type (endotype) characterized by chronic infiltration of eosinophils in the airway mucosa, often associated with increased severity, late-onset disease, and steroid refractoriness (Walford H H, Doherty T A: Diagnosis and management of eosinophilic asthma: a US perspective. J Asthma Allergy 2014, 7:53-65) (Shomali W, Gotlib J: World Health Organization-defined eosinophilic disorders: 2022 update on diagnosis, risk stratification, and management. Am J Hematol 2022, 97(1):129-148). Induced sputum differential cell count is the gold standard for identifying airway eosinophil-associated illnesses, while fractional exhaled nitric oxide (FeNO) and periostin are emerging as surrogates for disease progression. Elevated FeNO reflects active airway inflammation, correlating with increased disease severity and worsened asthma control. Additionally, FeNO is a non-invasive tool that predicts responsiveness to treatment and guides personalized asthma management.

Chronic rhinosinusitis with nasal polyps (CRSwNP) is a form of chronic sinonasal inflammation, with up to 60% of patients showing elevated eosinophils and associated proteins such as IL-5 and Eotaxin (e.g., Eotaxin-1, Eotaxin-2, Eotaxin-3, MCP-4) (J Allergy Clin Immunol Pract. 2016; 4(4): 565-572). The pathological conditions of CRSwNP are characterized by cosinophil-driven inflammation and impaired epithelial barriers, resulting in a dysregulated immune response. Despite current therapies, CRSwNP remains an unmet need due to its chronic and recurrent nature.

Eosinophilic esophagitis (EoE) is a chronic, immune-mediated disorder characterized by esophageal dysfunction and eosinophilic infiltration confirmed through biopsy, which shows at least 15 eosinophils per high-power field (Curr Opin Gastroenterol. 2012 July; 28(4): 382-388). The accumulation of eosinophils in the esophageal epithelium is the histopathologic hallmark. Despite current treatments, EoE remains a challenging condition with an unmet need for more effective therapies.

Type I interferons (IFN-α/β) have a critical role in regulating immune responses and protecting host against viral infection-induced asthma exacerbations (Pritchard A L, Carroll M L, Burel J G, White O J, Phipps S, Upham J W: Innate IFNs and plasmacytoid dendritic cells constrain Th2 cytokine responses to rhinovirus: a regulatory mechanism with relevance to asthma. J Immunol 2012, 188 (12): 5898-5905; Nakagome K, Nagata M: Innate Immune Responses by Respiratory Viruses, Including Rhinovirus, During Asthma Exacerbation. Front Immunol 2022, 13:865973.) In asthma patients, reduced IFN-α/β expression compromises epithelial integrity, contributing to eosinophilic-associated mucosal inflammation and disease progression (Heijink I H, Kuchibhotla V N S, Roffel M P, Maes T, Knight D A, Sayers I, Nawijn M C: Epithelial cell dysfunction, a major driver of asthma development. Allergy 2020, 75(8):1902-1917). Studies have demonstrated the anti-inflammatory role of IFN-β (Renatto Anfossi, Raúl Vivar, Pedro Ayala, et al: Interferon-β decreases LPS-induced neutrophil recruitment to cardiac fibroblasts. Front Cell Dev Biol 2023; 11:1122408). These mechanisms contribute to the understanding of IFN-I's role in respiratory infections. Additionally, up to 80% of asthma exacerbations are triggered by viral infections, IFN-I's modulation of various cytokines and negative regulation of eosinophils further underscores IFN-I's importance in managing airway inflammation (Johnston S L, Pattemore P K, Sanderson G, Smith S, Lampe F, Josephs L, Symington P, O'Toole S, Myint S H, Tyrrell D A et al.: Community study of role of viral infections in exacerbations of asthma in 9-11 year old children. BMJ 1995, 310(6989):1225-1229; Chang E Y, Guo B, Doyle S E, Cheng G: Cutting edge: involvement of the type I IFN production and signaling pathway in lipopolysaccharide-induced IL-10 production. J Immunol 2007, 178(11):6705-6709; Kotredes K P, Thomas B, Gamero A M: The Protective Role of Type I Interferons in the Gastrointestinal Tract. Front Immunol 2017, 8:410; Lee A J, Ashkar A A: The Dual Nature of Type I and Type II Interferons. Front Immunol 2018, 9:2061; Sousa J C, Etchbehere R M, Alves E A R, Stark L M, Murta E F C, Michelin M A: Interferon-alpha action in cytokine profile in eosinophilic nasal polyp cultures. Braz J Otorhinolaryngol 2021, 87 (3): 260-268).

The LTh(αK) has been developed into various products, including a nasal spray influenza vaccine (NCT03784885) and mucosal immunotherapy for SARS-CoV-2 (NCT05069610, NCT05541510), which demonstrates its versatility as a therapeutic agent.

### SUMMARY OF THE INVENTION

LTh(αK) is an immunomodulator that modulates innate, adaptive, and mucosal immunity while also reducing airway inflammation in patients with cosinophil-associated mucosal illnesses. Many chronic airway conditions linked to eosinophils, such as uncontrolled asthma, often resist or are refractory to corticosteroid therapy and can result in severe symptoms. Patients suffering from uncontrolled asthma frequently endure respiratory symptoms, including shortness of breath, chest pain or tightness, coughing, or wheezing, which disrupt their sleep and diminish the quality of life. In severe cases, uncontrolled asthma may lead to life-threatening asthma attacks.

An intranasal composition containing LTh(αK), which effectively treats eosinophil-associated airway mucosa illnesses, including uncontrolled asthma, eosinophilic chronic rhinosinusitis (eCRS), and eosinophilic esophagitis (EoE), has now been developed. Through intranasal administration, the composition reduces eosinophil counts in both blood and sputum. It decreases inflammation-associated biomarkers, thereby improving lung function in patients with these conditions and improving their quality of life. Additionally, LTh(αK) could reduce the dependency on inhaled corticosteroids (ICS) and bronchodilators, such as LABA or SABA. LTh(αK) represents a novel biological immunomodulator for the treatment of eosinophil-associated chronic airway inflammatory diseases.

LTh(αK) enhances the expression of type I interferons (IFN-I) by epithelial resident cells and nearby tissues. This induction of respiratory tract-restricted IFN-I has been demonstrated in both in vitro and in vivo studies. In vitro analyses demonstrated the detection of IFN-α in the medium of human primary bronchial epithelial cells cultured at an air-liquid interface. Additionally, a human clinical trial revealed elevated IFN-α levels in sputum after treatment with LTh(αK). These findings confirm that LTh(αK) activates IFN-I from the respiratory epithelium.

LTh(αK), through its ability to modulate immune activity has a pivotal role in controlling eosinophil-driven inflammation. Its intranasal or mucosal administration offers a targeted approach to reduce eosinophil counts and improve clinical outcomes in patients suffering from eosinophilic conditions, such as uncontrolled asthma, eCRS, and eosinophilic esophagitis (EoE).

The present invention is described in detail in the following sections. Further characterizations, purposes, and advantages of the invention can be found in the detailed descriptions and claims that follow.

### BRIEF DESCRIPTION OF THE FIGURES

FIG. 1 shows that LTh(αK) treatment on human primary bronchial epithelial cells (HBPECs) cultured in air-liquid interface (ALI) induces elevation of IFNα expression.

FIG. 2 shows that the LTh(αK)-induced IFN-α expression in ALI-cultured HBPECs was antagonized by pyrrolidine dithiocarbamate (PDTC), an inhibitor of NF-κB.

FIG. 3 shows that LTh(αK) intranasal administration enhances IFNα expression in the sputum of human asthmatic subjects compared to placebo.

FIGS. 4 and 5 show that LTh(αK), administered intranasally, reduces sputum and blood eosinophil counts compared to the placebo. The LTh(αK) and placebo were self-administered, twice a week, for a total of 11 doses. The final dose was administered on Day 36. *: End of Treatment. **: End of Study (Day 50).

FIG. 6 shows that LTh(αK) intranasal administration reduces sputum Eosinophilic Cationic Protein (ECP) levels compared to the placebo.

FIG. 7 shows that LTh(αK), administered intranasally, reduces the respiratory FeNO compared to the placebo. *: End of Treatment. **: End of Study.

FIGS. 8 and 9 show that LTh(αK) intranasal administration reduces blood and sputum IL-4 levels compared to the placebo. *: End of Treatment. **: End of Study.

FIG. 10 shows that LTh(αK), administered intranasally, reduces blood IL-5 compared to the placebo.

FIG. 11 shows that LTh(αK) treatment improves the Forced Expiratory Volume in 1 second (FEV1) compared to placebo.*: End of Treatment. **: End of Study.

### DETAILED DESCRIPTION OF THE INVENTION

Unless otherwise defined herein, scientific and technical terms used in connection with the present invention shall have the meaning commonly understood by those of ordinary skill in the art. The meaning and scope of the terms should be clear; however, in the event of any latent ambiguity, definitions provided herein take precedence over any dictionary or extrinsic definition.

As used in this disclosure, the following terms, unless otherwise indicated, shall be understood to have the following meanings:

Throughout this specification, the word “comprise” and variations such as “comprises” or “comprising” are understood to imply the inclusion of a stated integer (or components) or group of integers (or components), but not the exclusion of any other integer (or components) or group of integers (or components).

The singular forms “a,” “an,” and “the” are intended to include the plurals unless the context clearly dictates otherwise. Similarly, plural terms shall also include the singular unless otherwise required by the context.

“Treatment” and “treating” and the like refer to any approach to achieving a beneficial or desired outcome, including clinical outcomes. For purposes of this disclosure, such outcomes include but are not limited to, inhibiting or suppressing the onset or progression of a condition, reducing the severity of the condition, minimizing the number and/or intensity of symptoms, improving the quality of life for those affected, reducing the dosage of other medications required, enhancing the effects of another medication a patient is taking, and/or prolonging the survival of patients with the condition.

“Administering” or “administration of” a substance, a compound, or an agent to a subject can be carried out using one of a variety of methods known to those skilled in the art. For example, a compound or an agent can be administered intranasally. Administration can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another, and/or who provides a patient with a prescription for a drug is administering the drug to the patient.

The term “modulator” used herein refers to the agent that regulates a condition, level, or amount. The regulation may be upregulation or downregulation.

The terms “patient,” “subject,” or “individual” are used interchangeably and refer to either a human or a non-human animal. These terms include mammals, such as humans, primates, livestock animals (including bovines, porcine, etc.), companion animals (e.g., canines, felines, etc.), and rodents (e.g., mice and rats).

“Effective amount” refers to such amount of a therapeutic agent or a pharmaceutically acceptable salt thereof which, in consideration of its parameters of efficacy and potential for toxicity, as well as based on the knowledge of the practicing specialist, should be effective in a given therapeutic form. As is understood in the art, an effective amount can be administered in one or more doses.

The “anatomically or immunologically adjacent tissues” of the respiratory tissues refer to the tissues that eosinophils from the respiratory tissues may be present or migrate to. Examples of such tissues include, but not limited to, nasal tissues, oral tissues, throat tissues, trachea tissues, bronchi tissues, lung tissues, esophageal tissues, and ophthalmic tissues.

“Severe asthma” refers to asthma that requires Step 4-5 treatment (GINA Difficult-to-treat & severe asthma in adolescent and adult patients, V4.0 April 2023). Step 4-5 treatment involves high dose ICS-LABA. In this category, asthma symptoms are uncontrolled despite adherence with maximal optimized therapy and treatment of contributory factors or worsen when high-dose treatment is decreased.

“Eosinophilic chronic rhinosinusitis” (eCRS) refers to a chronic inflammatory condition of the nasal and paranasal sinus mucosa, characterized by significant eosinophil infiltration and immune responses driven by T-helper-2 cells. While eCRS can occur without nasal polyps, it shares the same eosinophilic-driven inflammation as CRSwNP. Eosinophils comprise a significant portion of the inflammatory cell population in eCRS and contribute to disease severity, frequent exacerbations, and impaired nasal function.

“Eosinophilic esophagitis” (EoE) is a chronic immune-mediated condition marked by eosinophilia inflammation, which is confined to the esophagus. Clinically, EoE presents with various esophageal dysfunctions, and pathologically, it is characterized by mucosal inflammation dominated by eosinophils. Although first identified in the early 1990s. EoE is now recognized as a major cause of digestive system disorders.

In the present invention, it was unexpectedly found that an intranasal composition comprising a genetically detoxified Escherichia coli heat-labile toxin (LT), such as LTh(αK), is effective in reducing eosinophil counts in both blood and sputum in patients with severe uncontrolled asthma. Accordingly, the present invention relates to a method for reducing eosinophils, which involves administering a therapeutically effective amount of a composition, wherein the composition comprises a genetically detoxified Escherichia coli heat-labile toxin (LT). In one aspect, the composition is for intranasal or mucosal administration.

In one embodiment, the detoxified LT is LTh(αK). LTh(αK) corresponds to LTS61K as disclosed in US 2008/0102078, which is a genetically detoxified E. coli LT holotoxin with a lysine substitution at the position corresponding to position 61 of SEQ ID NO: 5 as disclosed in US 2008/0102078.

In one embodiment, severe asthma is determined according to the Global Initiative for Asthma (GINA) 2023.

In one embodiment, severe asthma is determined according to the National Asthma Education and Prevention Program (NAEPP) Expert Panel Report (EPR)-4 2023.

In a preferred embodiment, severe asthma is uncontrolled, difficult to treat, or resistant to therapy.

In one embodiment, severe eosinophilic asthma involves excessive eosinophils detected from sputum or blood specimens. In a preferred embodiment, the patient has an absolute eosinophil count (AEC) from the blood of 500 eosinophils per microliter (μL). In a preferred embodiment, the patients/cosinophil count among induced sputum is higher than 3 percent of total sputum cells.

In one embodiment, the diagnostic criteria require three components for diagnosis of eosinophilic esophagitis: 1) Clinical symptoms of esophageal dysfunction; 2) a maximum esophageal eosinophil count of at least 15 cos/hpf, with few exceptions; and 3) exclusion of other possible causes of esophageal eosinophilia (Curr Opin Gastroenterol. 2012 July; 28(4): 382-388).

In one embodiment, the diagnosis criteria involve the presence of both subjective and objective evidence of chronic sinonasal inflammation. Symptoms include anterior or posterior rhinorrhea, nasal congestion, hyposmia, and/or facial pressure or pain lasting more than 12 weeks (J Allergy Clin Immunol Pract. 2016 July-August; 4(4): 565-572).

In one embodiment, the detoxified Escherichia coli labile toxin (LT) is LTh(αK).

In one embodiment, the detoxified Escherichia coli labile toxin (LT) is administered to an adult subject aged 20-80 years. In another embodiment, the subject has uncontrolled eosinophilic asthma. In another embodiment, the subject is diagnosed with asthma, and is followed up for a period of over two years. In one embodiment, the subject demonstrates post-bronchodilator reversibility of FEV₁ of ≥12% and ≥200 mL in response to a Short-Acting β2 Agonist (SABA). In another embodiment, the subject has more than 15% eosinophils in the induced sputum.

In one embodiment, the composition is administered intranasally or mucosally to the subject one or more times. In a preferred embodiment, the composition is administered once. In another preferred embodiment, the composition is administered twice. Alternatively, the composition may be administered multiple times. In one embodiment, the composition is administered to the subject upon occurrence of severe asthma symptoms.

In one embodiment, the administration of the composition induces Type I IFN production.

The present disclosure provides a method for treating elevated eosinophil levels in subjects with moderate to severe asthma, eosinophilic esophagitis (EoE), or chronic rhinosinusitis, by administering a therapeutically effective amount of a composition, wherein the composition comprises genetically detoxified Escherichia coli heat-labile toxin (LTh(αK)). In one aspect, the composition is for intranasal or mucosal administration. Unlike conventional treatments for moderate to severe asthma, EoE, and CRSwNP which often resulted in nonspecific attenuation to systemic immunity, the method of the present invention introduces a therapeutic effect confined to the respiratory system and related tissues. Moreover, the invention offers a safer treatment option for patients with elevated respiratory-associated eosinophils by providing an alternative therapy that maintains efficacy while reducing the risk of systemic side effects. The present invention facilitates the development of therapies with enhanced safety profiles for patients with elevated respiratory-associated eosinophil levels.

Having now generally described the invention, the same may be more readily understood through reference to the following examples. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed.

### EXAMPLES

### Example 1: In Vitro Studies for LTh(αK) Induced IFN-I Expression

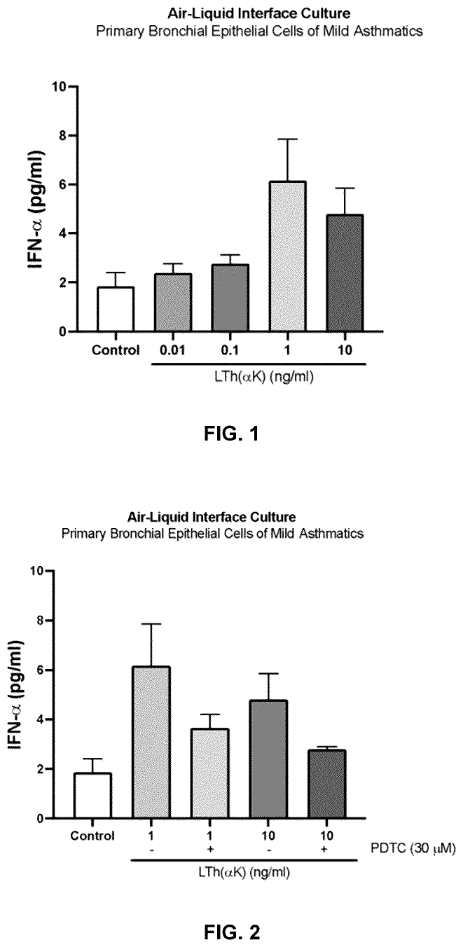

LTh(αK) treatment on human primary bronchial epithelial cells (HBPECs) induces expression of IFN-α. The HBPECs were collected from human subjects with mild asthma and cultured in the air-liquid interface (ALI) to promote differentiation. LTh(αK) treatment was initiated after HBPEC differentiation. The results show a treatment-induced elevation of IFN-α (FIG. 1). This expression was antagonized by pyrrolidine dithiocarbamate (PDTC), an inhibitor of NF-κB, a key factor in the IFN activation pathway (FIG. 2). The results demonstrate that LTh(αK) induces NF-κB-dependent IFN-α expression.

### Example 2: Human Study for LTh(αK) Treatment on Uncontrolled Moderate to Severe Asthmatic Adults with an Eosinophilic Phenotype

Asthma is characterized by chronic airway inflammation, which is linked to Th2 cytokine-mediated eosinophil infiltration of respiratory tissues, overproduction of mucus, hypersensitive bronchi, and overexpression of IgE. The real-world therapeutic data have proven pathological association between eosinophil and asthma (Lambrecht B N, Hammad H, Fahy J V: The Cytokines of Asthma. Immunity 2019, 50(4):975-991), and genetic evidence indicating loss of function of Illrl1 in humans leads to the absence of eosinophilia, and subsequent protection from asthma, which validates the pathological role of eosinophilia in asthma (Smith D, Helgason H, Sulem P, Bjornsdottir U S, Lim A C, Sveinbjornsson G, Hasegawa H, Brown M, Ketchem R R, Gavala M et al.: A rare IL33 loss-of-function mutation reduces blood eosinophil counts and protects from asthma. PLoS Genet 2017, 13(3):e1006659). Type I interferon, a cytokine expressed by epithelial cells at innate immune responses to LTh(αK), has anti-inflammatory properties and could antagonize eosinophilic activity within the respiratory compartment, leading to therapeutic effects in asthma (Sousa J C, Etchbehere R M, Alves E A R, Stark L M, Murta E F C, Michelin M A: Interferon-alpha action in cytokine profile in eosinophilic nasal polyp cultures. Braz J Otorhinolaryngol 2021, 87(3):260-268).

To assess the mechanisms and explore the potential efficacy of repeated LTh(αK) when given to patients with uncontrolled eosinophilic asthma, maintenance treatment of low to moderate-dose inhaled corticosteroids (ICS) and/or a combination with inhaled long-acting beta 2 agonist (LABA), plus short-acting beta-2-agonist (SABA) or ICS-formoterol may be administered as needed, to achieve better control over asthma.

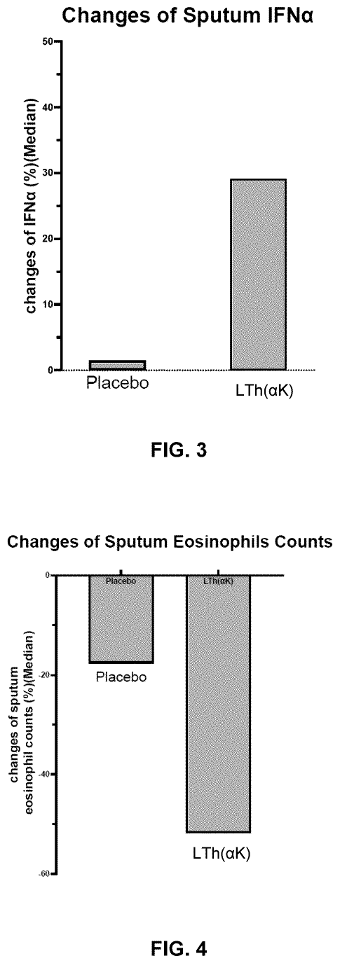

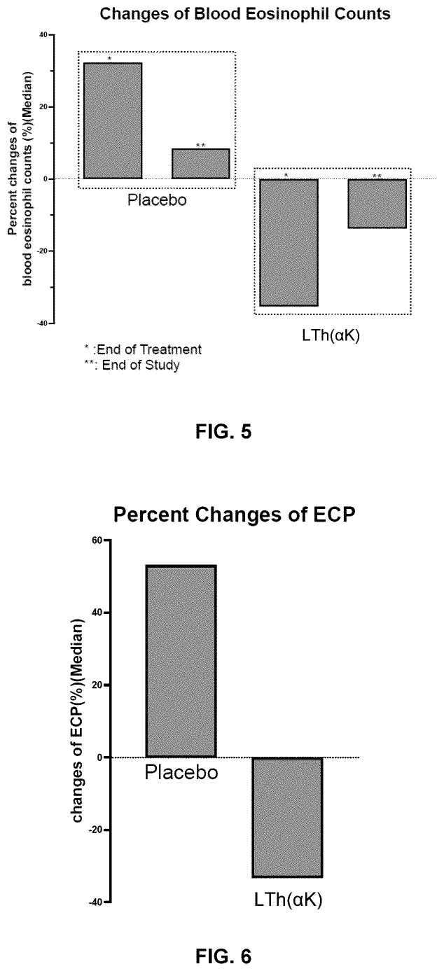

This is a single-center, single-blind (patient-blind), randomized, placebo-controlled, parallel study to assess the mechanisms and potential efficacy of intranasal 10 μg LTh(αK) every 3-4 days (twice a week) in modulating clinical responses in moderate to severe uncontrolled eosinophilic asthma (NCT05985694). This study demonstrated that intranasal LTh(αK) treatment induced sputum IFN-α (FIG. 3) and reduced sputum and blood eosinophil counts (FIGS. 4 and 5). Eosinophils are rarely detected in sputum among healthy individuals, and an increase in eosinophils in the respiratory tract is indicative of inflammation. The sputum eosinophils were collected on pre-dosing and end of study (EOS), that is, 2 weeks after the final LTh(αK) dose, and eosinophils were counted on the same day of specimen collection. In the LTh(αK) treatment group, patients exhibited reductions in sputum eosinophils by EOS compared to the placebo group (FIG. 4). Blood eosinophil counts were reported on end of treatment (EOT, last dosing day) (FIG. 5). The reduction of blood eosinophils was reported in patients who received LTh(αK) treatments but not in patients who had received placebo (FIG. 5).

Eosinophil cationic protein (ECP) is a potent mediator released by activated eosinophils. It plays a significant pathogenic role in eosinophilic asthma by inducing airway inflammation, epithelial damage, mucus hypersecretion, and airway hyperresponsiveness. In the LTh(αK) treatment group, patients exhibited reductions in sputum ECP by the EOS compared to the placebo treatment (FIG. 6).

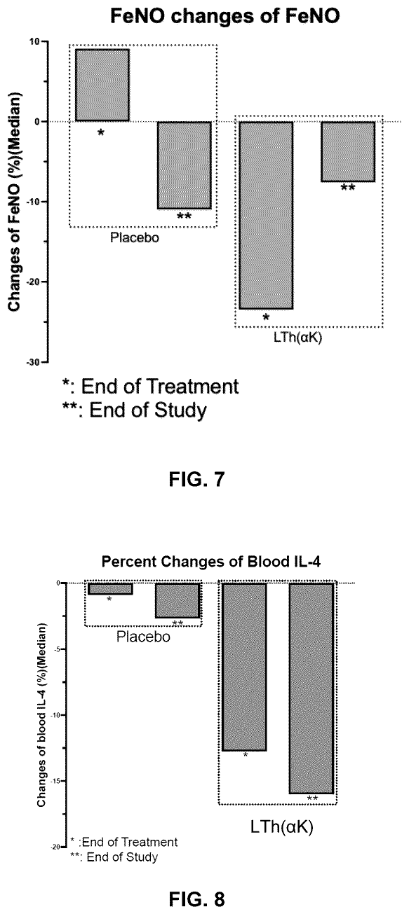

Fractional exhaled nitric oxide (FeNO) is an established biomarker indicative of airway inflammation. Clinically, elevated FeNO levels indicate active airway inflammation, which correlates with increased disease severity and poorer asthma control. Our LTh(αK) treatment demonstrated reductions in respiratory FeNO at the EOT (FIG. 7).

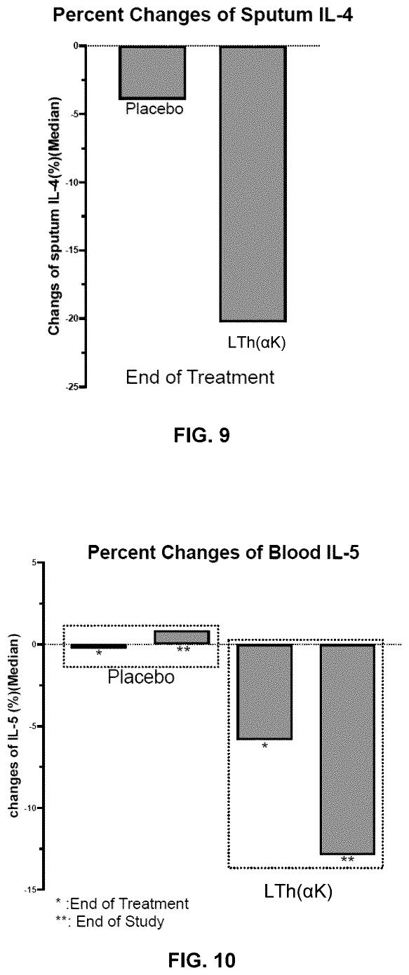

Interleukin-4 (IL-4) plays a pivotal role in asthma pathogenesis by driving type 2 inflammatory responses, including the production of IgE, the recruitment of inflammatory cells, and airway remodeling. Our data indicated that LTh(αK) treatment reduces IL-4 levels in both blood and sputum samples (FIGS. 8 and 9).

Interleukin-5 (IL-5) is a key cytokine in the pathogenesis of asthma, primarily regulating the differentiation, activation, and survival of eosinophils, which contribute to airway inflammation and hyperresponsiveness. Our data revealed that LTh(αK) treatment reduced blood IL-5 levels in EOT and EOS (FIG. 5).

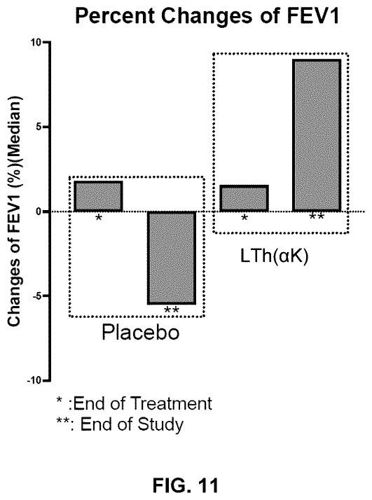

Forced expiratory volume in one second (FEV1) is a critical marker commonly used to evaluate lung function in asthma clinical trials, reflecting the degree of airway obstruction and the efficacy of treatment. An improvement in FEV1 indicates enhanced airflow, which is a central therapeutic goal in asthma management. Our clinical trial data demonstrated a trend of improvement in FEV1 following treatment of LTh(αK) at EOS (FIG. 11).

What is claimed is:

1. A method for treating a subject with eosinophil-associated inflammation in respiratory tissues and anatomically or immunologically adjacent tissues, comprising administering to the subject a therapeutically effective amount of a composition comprising detoxified Escherichia coli heat-labile toxin (LT), wherein the composition is administered via a mucosal route.

2. The method of claim 1, wherein the composition is administered via an intranasal route.

3. The method of claim 1, wherein the eosinophil-associated inflammation comprises asthma.

4. The method of claim 1, wherein the eosinophil-associated inflammation comprises eosinophilic chronic rhinosinusitis (eCRS).

5. The method of claim 1, wherein the eosinophil-associated inflammation comprises eosinophilic esophagitis (EoE).

6. The method of claim 3, wherein the asthma is moderate to severe asthma, determined according to the Global Initiative for Asthma (GINA) 2023.

7. The method of claim 3, wherein the asthma is moderate to severe asthma determined according to the National Asthma Education and Prevention Program (NAEPP) Expert Panel Report (EPR)-4 2023.

8. The method of claim 1, wherein the detoxified Escherichia coli heat-labile toxin (LT) is LTh(αK).

9. The method of claim 1, wherein the composition is administered one or more times.

10. The method of claim 1, wherein the composition is administered alone.

11. The method of claim 1, wherein the composition is administered in combination with other treatments.

12. The method of claim 1, wherein administration of the composition induces Type I interferon (IFN) production.

13. The method of claim 1, wherein administration of the composition reduces the blood eosinophil counts.

14. The method of claim 1, wherein administration of the composition reduces the sputum eosinophil counts.

15. The method of claim 1, wherein administration of the composition reduces the fractional exhaled nitric oxide (FeNO).

16. The method of claim 1, wherein administration of the composition reduces the sputum eosinophilic cationic protein (ECP).

17. The method of claim 1, wherein administration of the composition reduces the blood IL-4 cytokine.

18. The method of claim 1, wherein administration of the composition reduces the sputum IL-4 cytokine.

19. The method of claim 1, wherein administration of the composition reduces the blood IL-5 cytokine.

20. The method of claim 1, wherein administration of the composition enhances the Forced Expiratory Volume in 1 second (FEV1).
